(19) United States
(12) Patent Application Publication
(10) Pub. No.: US 2013/0090650 A1
(43) Pub. Date: Apr. 11, 2013

(54) RENAL NERVE ABLATION COOLING DEVICE AND TECHNIQUE

(71) Applicant: Boston Scientific Scimed, Inc., Maple Grove, MN (US)

(72) Inventors: MARK L. JENSON, GREENFIELD, MN (US); TRAVIS J. SCHAUER, DELANO, MN (US); SCOTT R. SMITH, CHASKA, MN (US)

(73) Assignee: BOSTON SCIENTIFIC SCIMED, INC., MAPLE GROVE, MN (US)

(21) Appl. No.: 13/647,146

(22) Filed: Oct. 8, 2012

Related U.S. Application Data
(60) Provisional application No. 61/545,950, filed on Oct. 11, 2011.

(51) Int. Cl. A61B 18/14

(52) U.S. Cl. 606/41

(57) ABSTRACT

A catheter is disclosed including an elongated shaft having a distal end and a proximal end, where the catheter includes a thermal element at the distal end thereof. The thermal element may be used in an ablation procedure or other procedure to heat a tissue adjacent a vessel. The configuration of the distal end of the elongated shaft at or near the distal tip may encourage the cooling of or transferring of heat from the vessel wall. The configurations may include protrusions extending from and indentations extending into the shaft, which may manipulate the flow of fluid through a vessel in which the catheter has been inserted. Alternatively or additionally, a cap or thin insulative layer may be placed at or near the distal tip of the catheter shaft to cool the wall of the vessel.

### CROSS-REFERENCE TO RELATED APPLICATIONS

This application claims priority under 35 U.S.C. §119 to U.S. Provisional Application Ser. No. 61/545,950, filed Oct. 11, 2011, the entirety of which is incorporated herein by reference.

### TECHNICAL FIELD

The disclosure is directed to catheters for insertion into bodily vessels. More particularly, the disclosure is directed to catheters for use in neuromodulation procedures, such as renal denervation procedures.

### BACKGROUND

Conventional catheters are used in medical procedures to gain access to interior regions of bodies. An illustrative region of a body in which catheters are often used is in the cardiovascular system. Typically, a catheter for insertion into a body may have a distal end for insertion into an interior of the body and a proximal end that remains exterior to the body. Catheters may be used in a variety of medical procedures including, but not limited to ablation procedures, angioplasty procedures, therapeutic procedures, diagnostic procedures and exploratory procedures, among others.

### SUMMARY

The disclosure is directed to several alternative or complementary designs, materials and methods of using medical device structures and assemblies. Although it is noted that conventional catheters exist, there exists need for improvement on those devices.

Accordingly, one illustrative embodiment of the disclosure may include a catheter having an elongated shaft with a distal end and a proximal end at opposing ends thereof. The distal end of the elongated shaft may include a thermal element that may be used for heating and/or ablating a tissue adjacent a vessel wall in which the catheter has been inserted through the use of an energy field or other technique. In addition, the distal end of the catheter may be configured to facilitate heat transfer away from the vessel wall (e.g., cooling the vessel wall) when the catheter is being utilized to heat and/or ablate a perivascular tissue. The heat transferred away from the vessel wall may be transferred to a fluid flowing through the vessel in which the catheter has been inserted and/or directly into the catheter. To achieve and/or encourage heat transfer from the vessel wall to the flowing fluid and/or the catheter, the catheter may be configured to modify the flow of fluid flowing through the vessel. For example, the flow of fluid flowing through the vessel may be modified by utilizing and configuring materials of the elongated shaft and thermal element, and the placement and shapes of those materials, to reduce contact between the catheter and vessel wall, reduce sizes of hot zones within or near the vessel wall and/or reduce thicknesses of boundary layers of fluid flowing through the vessel.

The above summary of some example aspects is not intended to describe each disclosed embodiment or every implementation of the claimed disclosure.

### BRIEF DESCRIPTION OF THE DRAWINGS

The disclosure may be more completely understood in consideration of the following detailed description of various embodiments in connection with the accompanying drawings, in which:

FIG. 1 is a schematic side view of an existing catheter apparatus inserted into a vessel;

FIG. 2 is a schematic side view of a catheter apparatus inserted into a vessel according to an aspect of the disclosure;

FIG. 3 is a schematic side view of a catheter apparatus inserted into a vessel according to an aspect of the disclosure;

FIG. 4 is a schematic side view of a catheter apparatus inserted into a vessel according to an aspect of the disclosure;

FIG. 5 is a schematic side view of a catheter apparatus inserted into a vessel according to an aspect of the disclosure;

FIG. 6A is a schematic side view of a catheter apparatus inserted into a vessel according to an aspect of the disclosure;

FIG. 6B is a schematic cross-sectional view of the catheter apparatus of FIG. 6A taken along line 6B-6B;

FIG. 7A is a schematic side view of a catheter apparatus inserted into a vessel according to an aspect of the disclosure;

FIG. 7B is a schematic cross-sectional view of the catheter apparatus of FIG. 7A taken along line 7B-7B;

FIG. 8A is a schematic side view of a catheter apparatus inserted into a vessel according to an aspect of the disclosure;

FIG. 8B is a schematic cross-sectional view of the catheter apparatus of FIG. 8A taken along line 8B-8B;

FIG. 9A is a schematic side view of a catheter apparatus inserted into a vessel according to an aspect of the disclosure;

FIG. 9B is a schematic cross-sectional view of the catheter apparatus of FIG. 9A taken along line 9B-9B;

FIG. 10A is a schematic magnified side view of features of a catheter apparatus according to an aspect of the disclosure;

FIG. 10B is a schematic cross-sectional view of the features of the catheter apparatus of FIG. 10A taken along line 10B-10B;

FIG. 11A is a schematic side view of a catheter apparatus inserted into a vessel according to an aspect of the disclosure; and

FIG. 11B is a schematic cross-sectional view of the catheter apparatus of FIG. 11A taken along line 11B-11B.

While the disclosure is amenable to various modifications and alternative forms, specifics thereof have been shown by way of example in the drawings and will be described in detail. It should be understood, however, that the intention is not to limit aspects of the claimed disclosure to the particular embodiments described. On the contrary, the intention is to cover all modifications, equivalents, and alternatives falling within the spirit and scope of the claimed disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the claimed disclosure. The illustrative embodiments depicted are intended only as exemplary. Selected features of any illustrative embodiment may be incorporated into an additional embodiment unless clearly stated to the contrary.

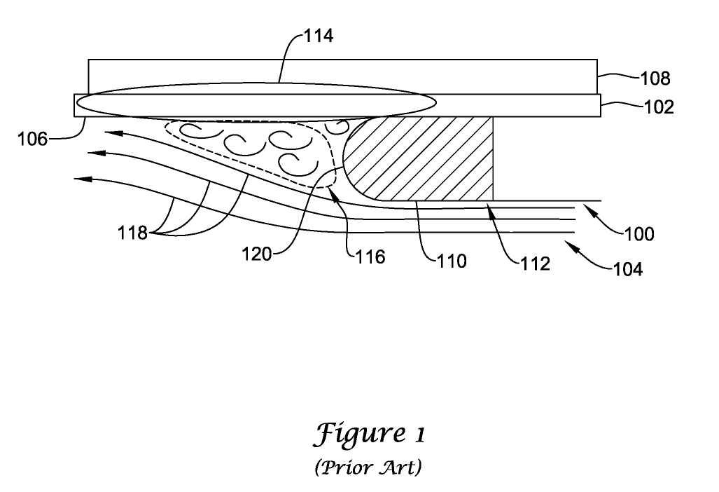

Generally, techniques for cooling a vessel 102 during an ablation procedure have included passive cooling (e.g., through the flow of blood in the renal artery), or active cooling (e.g., infusion into the vessel of a cooling fluid or other cooling technique). It may be known that active cooling has been more effective at preventing injury to vessels 102 due to heat than passive cooling, but active cooling may also be known to be clumsy to administer. An illustrative passive cooling technique, as seen in FIG. 1, may be used to cool vessel 102 during an ablation procedure. In the illustrative technique, an ablation catheter 100 may be inserted into a lumen 104 of a vessel 102 adjacent an ablation target tissue 108 (e.g., a perivascular tissue). A thermal element 110 configured to generate radiant heat (e.g., a radio-frequency (RF) electrode, ultrasound transducer, etc.) may be located at a distal end 112 of catheter 100 may be positioned at or near vessel wall 106 to ablate material from or the material of a tissue 108 adjacent vessel 102. For example, when catheter 100 has been placed in contact with vessel wall 106 (e.g., the wall of a renal artery), catheter 100 may be used to ablate (e.g., denervate) perivascular renal nerves (e.g., renal sympathetic nerves) or other tissue 108 adjacent or near vessel 102 to treat or alleviate hypertension, heart failure, chronic kidney diseases and other vascular related issues. During this process, wall 106 of lumen 104 may be heated and the flow 118, such as a laminar flow of fluid through vessel 102 may be used to passively remove heat from vessel wall 106.

During neuromodulation or denervation procedures, catheter 100 may abut vessel wall 106 and a zone 114 (see circled area 114 in FIG. 1) of high temperature may form in, on or near vessel wall 106 at and/or distal or downstream to distal end 112 of catheter 100, as seen in FIG. 1. It has been determined that during some procedures vessel wall 106 may experience an increase in temperature approaching 63° C. above a normal body temperature, which may be near the boiling point of water (e.g., 37° (˜body temperature) +63° C. (˜increase in temperature)=100° C. (˜boiling point of water)), in zone 114 consequent thermal energy passing into vessel wall 106 from a thermal element 110 during an ablation procedure. Whereas, a therapeutic rise in temperature may be between 10-30° C. in zone 114 consequent thermal energy passing into vessel wall 106. Generally, zone 114 may result from laminar flow 118 (as indicated by streamline arrows in FIG. 1) in vessel 102 creating an area of flow separation, or a separated region, such as an eddy or eddies 116 just distal of distal tip 120 at a position near where thermal element 110 actively heats tissue 108. Typically, lower velocities, less mixing and less heat transfer are present in this area of flow separation compared to other portions of the vessel lumen 104. Due to the size of eddies 116 at or near distal end 112 of catheter 100 and the low fluid velocities therein, there may be poor fluid mixing distal or downstream of distal end 112 and heat may be insufficiently transferred from vessel wall 106 and eddy 116 to the rest of the fluid in vessel 102. Fluid in eddy 116 may experience heating from energy radiated from thermal element 110 of catheter 100, in addition to receiving heat from wall 106. Thus, because of eddy 116 and the resultant flow separation, the heat may not be passed away from wall 106 as effectively or efficiently as may be desired to prevent overheating and damage to wall 106. As such, zone 114 may be considered undesirable because it may cause a thermal injury (e.g., a burn, stenosis, etc.) or other injury to vessel 102. Although active cooling techniques may be used to mitigate heat damage to the vessel, these techniques have been found to be inconvenient and not to address the issue of hot zones 114. Thus, fluid passing through vessel 102, by itself, may provide insufficient cooling to vessel 102 during a typical ablation procedure and as a result, known ablation techniques or approaches have not been effective at preventing thermal injury to vessel walls 106.

As described herein, a catheter 10, including a thermal element 30, may be used for any purpose and may be considered an ablation catheter 10 or another type of catheter 10. Ablation catheter 10 may be configured to be inserted into a vessel 12 to remove and/or affect material of a tissue 18 adjacent vessel 12. For example, ablation catheter 10 may be configured to be inserted into a renal artery to denervate or ablate (e.g., remove or affect) perivascular renal nerves adjacent the renal artery.

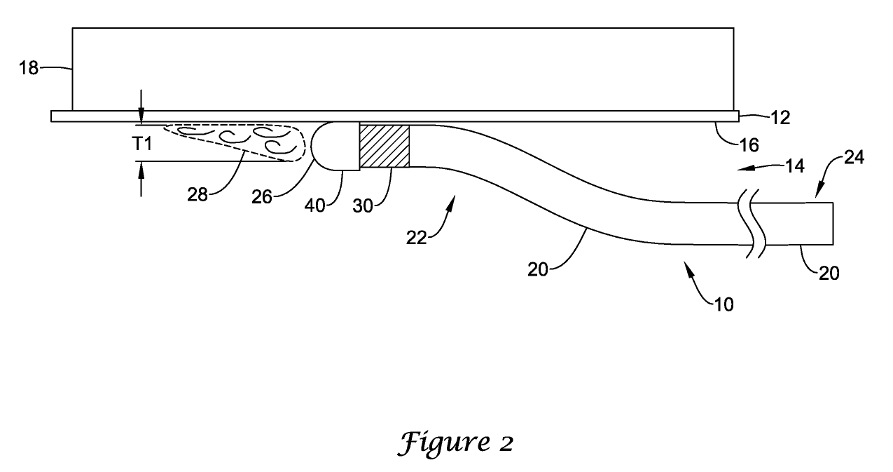

Referring to FIGS. 2-11B, catheter 10 for insertion into a lumen 14 of vessel 12 is shown. Catheter 10 may have an elongated shaft 20 having a distal end 22 and a proximal end 24 at opposing ends thereof. In addition, distal end 22 may have a distal tip 26 which may be defined as the material at or adjacent a terminal end thereof. In an illustrative instance, distal end 22 may be intended to be at least partially inserted into an interior of vessel 12 and proximal end 24 may be intended to be at least partially exterior of vessel 12. Further, distal end 22 of elongated shaft 20 may be configured to facilitate heat transfer away from vessel wall 16 and into elongated shaft 20 and/or into a fluid flowing through vessel 12 to cool vessel wall 16 or for other purposes before, after or during the ablation procedure, for example.

Among other features, catheter 10 may include thermal element 30. Thermal element 30 may be located on or in catheter 10 at or near distal end 22 and upstream or proximal of distal tip 26 of catheter 10 and may be an electrode or device configured to heat a tissue adjacent vessel wall 16 through an energy field using radio-frequency heating or ultrasound heating or another heating technique. Generally, thermal element 30 of catheter 10 may be any size. For example, thermal element 30 may be 2-4 millimeters in length, including an insulation portion, and 1.5 millimeters in diameter or height, or thermal element 30 may have other dimensions. The insulation portion may include a cap 40 or a thin layer of material applied to shaft 20 and thermal element 30 may be increased in length to keep current density emitted therefrom within acceptable predetermined ranges or limits.

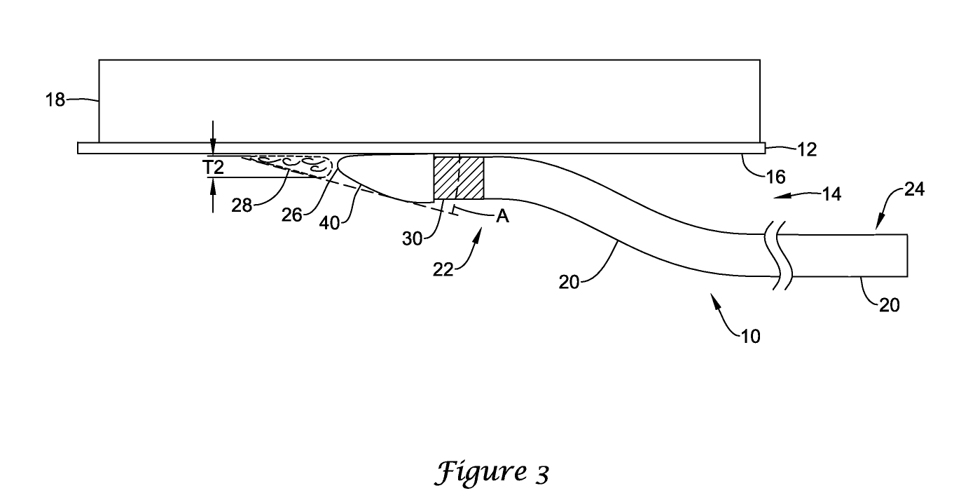

A catheter 10 that may generally be used in ablation or denervation procedures may include an elongated shaft 20 made out of a polymer or other suitable material configured to be inserted into vessel 12 and capable of physical manipulation throughout a vascular system. In addition, elongated shaft 20 may include thermal element 30, which may be an electrode (e.g., a cylindrical radio-frequency (RF) ablation electrode) or other device that facilitates RF heating or ultrasound heating or other similar or different types of heating using an energy field. As mentioned, thermal element 30 may be placed at a position on elongated shaft 20 proximate distal end 22. For example, thermal element 30 may extend from distal tip 26 or from a position proximal of distal tip 26 and may extend in a proximal direction with respect to distal tip 26, as seen in at least FIGS. 2 and 3.

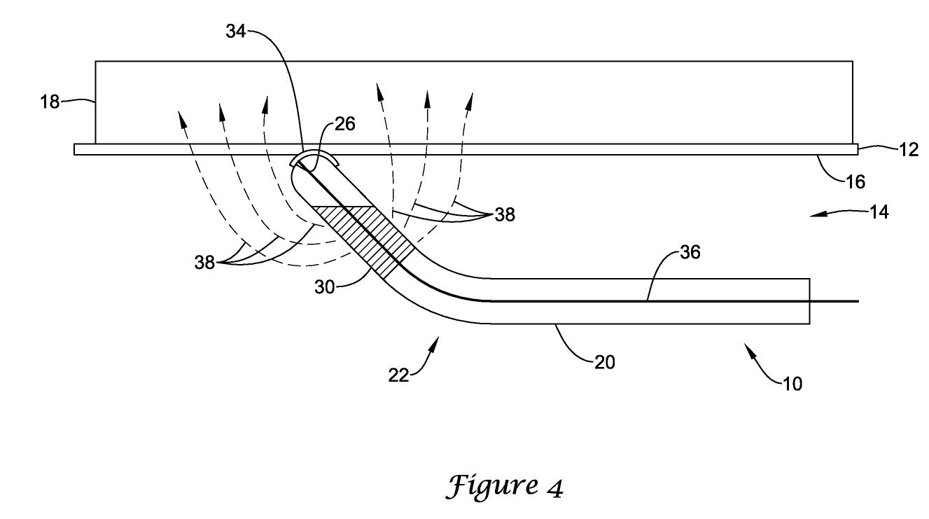

Additionally or alternatively, an illustrative catheter 10 may include a sensor 34 formed along or at an exterior of elongated shaft 20 and at or near distal tip 26 and downstream of thermal element 30, as seen in FIG. 4. Sensor 34 may be any type of sensor. For example, sensor 34 may be a temperature and/or impedance sensor and/or any other type of sensor configured to monitor the ablation procedure. In the example, sensor 34 may monitor the ablation procedure by measuring blood temperature downstream of thermal element 30 and provide an early indication of overheating, in addition to monitoring other similar or different characteristics affected by the ablation procedure.

Sensor 34 may include a sensor wire or wires 36 extending from sensor 34 through elongated shaft 20 to a user interface (not shown). Where elongated shaft 20 may extend distal of thermal element 30, or there may be an insulating portion (e.g., a cap 40 or a thin layer of material) of elongated shaft 20 distal or downstream of thermal element 30, or the insulating portion may cover a portion of thermal element 30 adjacent distal tip 26, or thermal element 30 adjacent distal tip 26 may be uncovered or covered by a non-insulating portion, catheter 10 may be manipulated such that sensor 34 may be placed or positioned in contact with vessel wall 16 through active (e.g., operator manipulated deflection) or passive (e.g., pre-determined deflection) deflection of catheter 10. In this illustrative example, RF waves 38 may enter tissue 18 at positions other than where elongated shaft 20 may contact vessel wall 16, as seen in FIG. 4. Generally, RF waves 38 may provide the most heat per unit volume at or near thermal element 30 and the heat generated by RF waves 38 may decay proportionally to 1/r⁴, where r=the distance from thermal element 30. Through these disclosed techniques and others, sensor 34 may abut vessel wall 16 while thermal element 30 may be held off of wall 16 or kept from direct contact with wall 16. This arrangement or technique may facilitate denervating or ablating a target tissue without direct contact between thermal element 30 and wall 16 and thus, allows for passive cooling (e.g., via fluid flowing through vessel 12) between thermal element 30 and wall 16, while sensor 34 may monitor tissue and/or blood temperatures downstream of thermal element 30 to safely and/or efficiently control the procedure and monitor the health of wall 16 before, during and/or after a procedure with catheter 10. In addition, this technique may allow for catheter 10 to act as a heat sink pulling heat from vessel wall 16.

As seen in FIGS. 2 and 3, a cap 40 may be placed on distal end 22 of elongated shaft 20 to facilitate heat transfer away from vessel wall 16 or for other purposes. Cap 40 may at least partially cover distal tip 26 at distal end 22 of elongated shaft 20 and may provide a buffer between thermal element 30 and vessel wall 16. In some instances, cap 40 may circumferentially cover a portion of elongated shaft 20 at or near distal tip 26 and may or may not extend over a portion of thermal element 30. Cap 40 may be a piece formed separately from elongated shaft 20 that may be placed onto distal end 22 of shaft 20; cap 40 may be formed as part of elongated shaft 20 and may be an added layer thereto at or near distal end 22; cap 40 may be a layer (e.g., a relatively thin layer compared to the thickness of catheter 10) or particles of a material applied to a portion of distal end 22 of elongated shaft 20; or cap 40 may take on another form. Cap 40 may prevent excess heating of vessel wall 16 proximate the distal terminus of catheter tip. For example, cap 40 may position thermal element 30 away from eddy zone 28 and/or away from vessel wall 16.

Further, cap 40 or the thin insulating layer may be made from any material that may be electrically insulating with a high electrical impedance (e.g., that may be electrically non-conductive), yet thermally conductive with a low thermal resistance (e.g., thermally non-insulating). Illustratively, the material of cap 40 or the thin insulating layer may be a diamond-like carbon (DLC), parylene or other chemical vapor deposited poly(p-xylylene polymers, a ceramic material (e.g., aluminum oxide), highly filled polymers (e.g., polymers filled with metal or metal oxide), silicone or other similar polymers or other material having similar properties. Such material may be capable of facilitating heat transfer into distal end 22 of catheter with reduced radio-frequency (RF) heating needed to heat tissue 18. The material of cap 40 may be the same or different than the material of elongated shaft 20 (e.g., a polymer material or a different, but known, material for ablation catheters). Illustratively, the material and design of cap 40 may allow cap 40 to act as a heat sink removing heat from vessel wall 16 and transferring it to a fluid flowing through vessel 12 or to elongated shaft 20 to cool vessel wall 16, while preventing thermal element 30 from directly heating and/or preventing RF energy of thermal element 30 from passing directly to vessel wall 16 at a location immediately distal of distal tip 26 and/or directly abutting vessel wall 16. Further, cap 40 may prevent RF energy from passing directly from thermal element 30 to vessel wall 16.

Cap 40 may take on any shape or size configured to facilitate transferring heat from vessel wall 16 or cooling vessel wall 16. For example, a portion of distal end 22 of elongated shaft 20 may taper toward distal tip 26 and cap 40 may be similarly tapered, as seen in FIG. 3. Alternatively, cap 40 may be tapered without distal tip 26 having a taper. The taper of cap 40 may be asymmetrical where one portion of the taper tapers more than the other portion (e.g., a tapering ellipse shape), as seen in FIG. 3, or the taper may be symmetrical (e.g., cone-shaped). Further, the taper may take on any desirable angle A with respect to wall 16. In addition, cap 40 and/or the thin layer of material may have any desired thickness. For example, cap 40 and/or the thin layer of material may have a thickness of about 100 nanometers, about 200 nanometers, about 500 nanometers, about 1 micron, about 10 microns, about 20 microns, about 100 microns or any other desirable thickness. In the example, the thicknesses of cap 40 and the thin layer from elongated shaft to an exterior of cap 40 or the thin layer may be in the range of about 100 nanometers to about 10 microns.

In addition to cap 40 acting as a heat sink and drawing heat from vessel wall 16 and an eddy zone 28 distal of distal tip 26, the shape of cap 40 may facilitate cooling vessel wall 16 adjacent distal tip 26 in other manners. For example, an asymmetrical taper of cap 40 may manipulate a flow, such as a laminar flow of fluid through vessel 12 in such a manner as to reduce the size of eddy zone 28 such that it may have a maximum thickness of T2 (see FIG. 3), where eddy zone 28 of a distal end 22 without a taper may have a maximum thickness T1 (see FIG. 2) that is greater than thickness T2. As a result, the taper in cap 40 or distal end 22 may create a smaller, if any, volume 28 of fluid not being consistently carried away by the fluid flowing through vessel 12 than when there is no taper in cap 40 or distal end 22. A small eddy zone 28 may result in more effective heat transfer than in a large eddy zone 28 because the cooler fluid flowing through vessel 12 passes closer to vessel wall 16 such that there may be a smaller volume of fluid in the smaller eddy zone 28 to be cooled than there may be in a larger volume eddy zone 28.

In addition to the shape and other features of cap 40 facilitating the cooling of vessel wall 16, cap 40 may facilitate such cooling by creating a space between thermal element 30 and vessel wall 16. As seen in FIGS. 2 and 3, catheter 10 may be utilized by placing cap 40 adjacent wall 16 and leaving a space between vessel wall 16 and thermal element 30, which may allow fluid to flow in the space and facilitate heat transfer away from vessel wall 16. Otherwise, if thermal element 30 were to directly abut vessel wall 16, the portion of vessel wall 16 in direct contact with thermal element 30 may be blocked from being cooled by fluid flowing through vessel 12 or at least prevented from being cooled as efficiently as possible through an indirect heating technique where thermal element 30 may not be in direct contact with vessel wall 16.

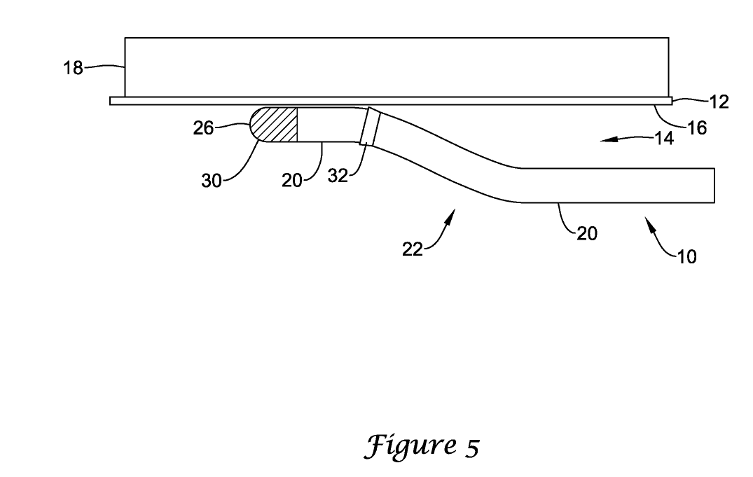

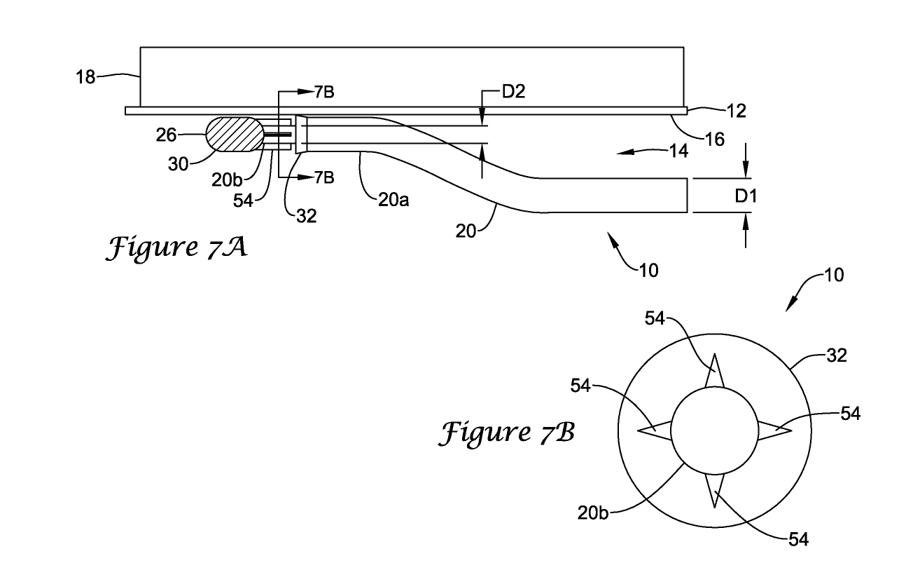

Catheter 10 may also include a trip feature 32 on or extending from elongated shaft 20, as seen in FIGS. 5 and 7A, where trip feature 32 may be a balloon, a trip wire, annular rim, flare, ledge or other device or structure that an operator may introduce at a desirable time or moment or that may be present without prompting by an operator. Trip feature 32 may be located upstream or proximal of thermal element 30 and may continuously and circumferentially extend around elongated shaft 20 or may take on various forms at least partially circumferentially spaced about elongated shaft 20. Trip feature(s) 32 may assist in heat transfer away from, or the cooling of, vessel wall 16 by changing the boundary layer characteristics of the blood flowing through lumen 14, such as creating disturbed and/or turbulent boundary layers in fluid flowing through vessel 12 or by other similar or different means, and/or reducing the size of the separated flow region downstream or distal of the distal end of the catheter 10. For example, trip features 32 (e.g., annular rim, balloons or other devices) may be configured to increase the velocity of blood flowing near the wall 16 of vessel 12 relative to without the trip features 32. In some instances, trip features 32 may create a disturbed or turbulent boundary layer. It is noted that at a given Reynolds number, even though a turbulent boundary layer may actually be thicker than a laminar boundary layer, the turbulent boundary layer promotes better heat transfer away from the vessel wall 16 due to the velocity gradient and enhanced mixing in the turbulent boundary layer. Thus, creating a disturbed or turbulent boundary layer of fluid flowing through vessel 12 may facilitate or promote heat transfer away from, or the cooling of, vessel wall 16 at positions downstream of thermal element 30 and distal tip 26 of elongated shaft 20 and/or at positions adjacent trip feature 32 because of the velocity gradient and enhanced mixing of the fluid in the transitional or disturbed or turbulent boundary layer. In addition, a disturbed or turbulent boundary layer may promote or facilitate heat transfer away from, or the cooling of, vessel wall 16 due to its ability to stay attached to a blunt shape (e.g., catheter 10) for longer than a laminar boundary layer, thus reducing the volume or size of a separated flow region or eddy zone 28 downstream of distal tip 26. Also in the example, trip features 32 may be made of a material having thermally conductive and electrically insulating properties or any other material having similar or different properties. In addition to trip feature 32, active cooling techniques discussed above may be used in combination with other cooling techniques to encourage heat transfer away from vessel wall 16 and/or to cool vessel wall 16.

As alluded to above, the thickness of the boundary layers may be related to a type of flow of the fluid. For example, a laminar flow may have thin boundary layers, whereas a more turbulent flow may have thicker boundary layers and a transitional flow may have boundary layers with thicknesses between that of a laminar flow and a turbulent flow. A Reynolds number is a unit of measurement generally used to determine the type of flow and techniques for determining a Reynolds number of a flowing fluid are well known. Illustrative Reynolds numbers (Re) for flows in a pipe (e.g., a vessel) of diameter D may include a Re of less than 2,300 for a laminar flow, a Re between 2,300 and 4,000 for a transitional or disturbed flow (e.g., a mix of laminar and turbulent flows may be possible), and a Re above 4000 for a turbulent flow. Further, flow characteristics of disturbed and turbulent flows may have similarities; however, disturbed boundary layers may redevelop into a laminar boundary layer as fluid flows downstream, whereas turbulent flows may remain turbulent as fluid flows downstream.

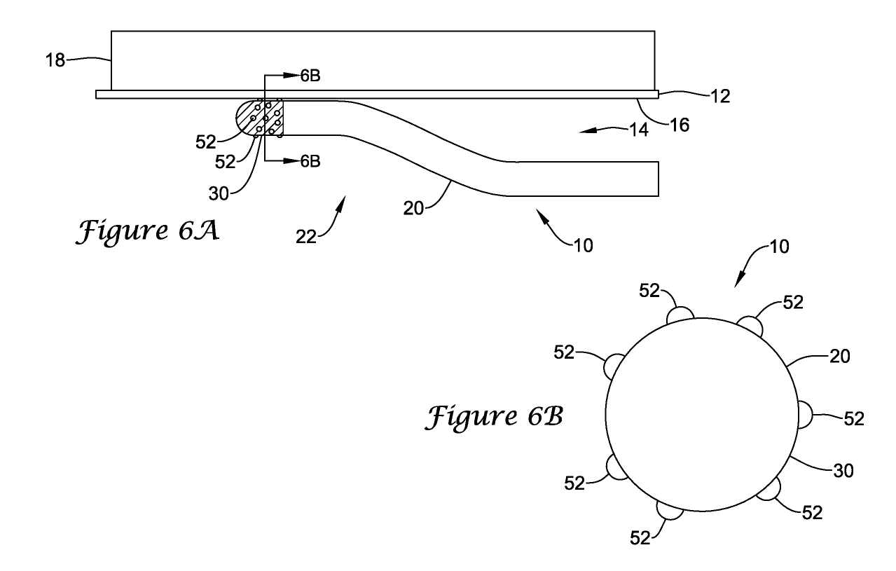

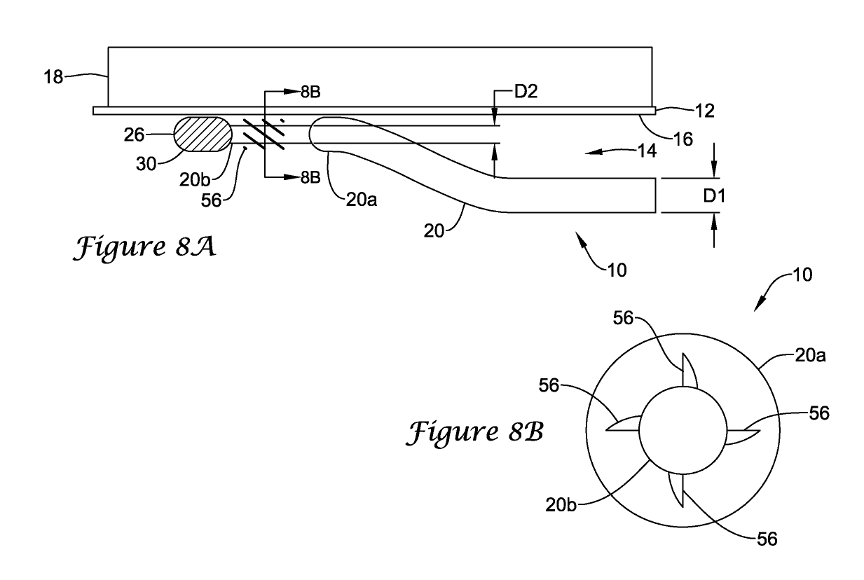

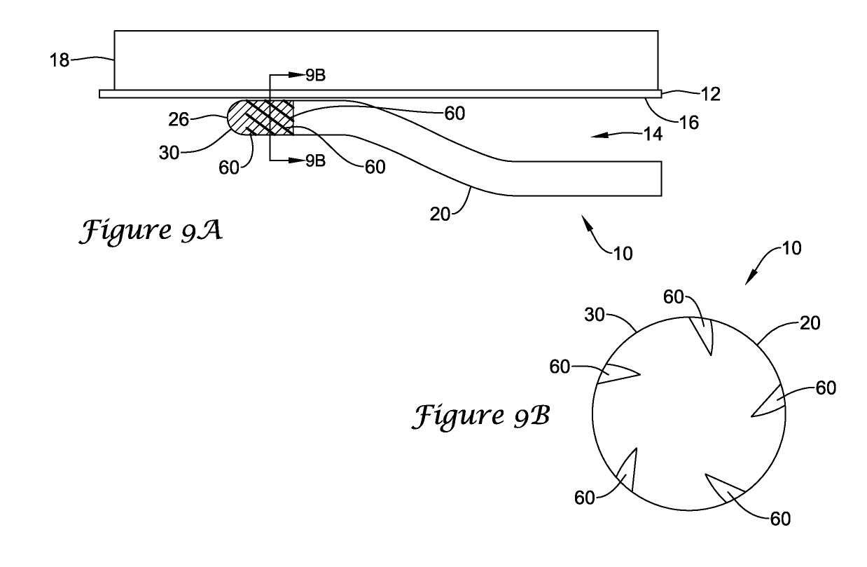

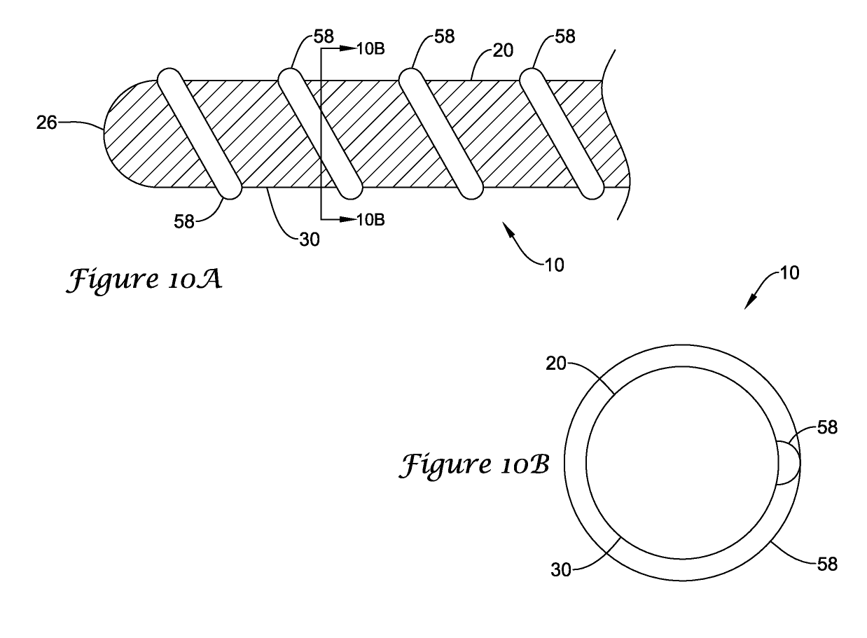

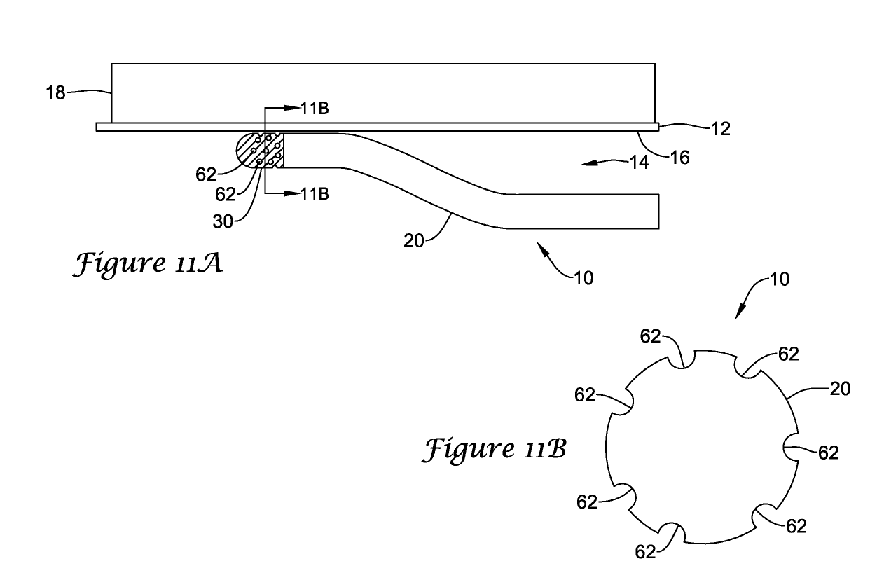

In addition to trip features 32 of catheter 10 causing disturbed flows to alter, disrupt or disturb boundary layers of the flow (e.g., create a turbulent boundary layer) and facilitate heat transfer away from vessel wall 16, distal end 22 may be further configured to create a disturbed or turbulent flow of fluid flowing through vessel 12 at positions near or adjacent, or distal of distal tip 26 of elongated shaft 20. For example, elongated shaft 20 or cap 40, or a feature of catheter 10 extending from elongated shaft 20, may include one or more protrusions, one or more bumps 52 (FIGS. 6A and 6B), one or more fins 54 (FIGS. 7A and 7B), one or more vanes 56 (FIGS. 8A and 8B), one or more spiral channels 60 (FIGS. 9A and 9B), one or more spiral protrusions 58 (FIGS. 10A and 10B), one or more dimples or indentations 62 (FIGS. 11A and 11B) and/or one or more other features that may disrupt a flow of a fluid flowing through vessel 12.

In an illustrative instance, elongated shaft 20 may have bump(s) 52 extending from and/or indentation(s) or dimple(s) 62 extending into distal end 22. For example, bumps 52 and dimples 62 may directly extend from or into thermal element 30 as seen in FIGS. 6A. and 11A. Although bump(s) 52 and dimple(s) 62 may be shown extending from or into thermal element 30, bump(s) 52 and dimple(s) 62 may directly or indirectly extend from or into, respectively, thermal element 30 and/or elongated shaft 20 and/or cap 40. Bump(s) 52 and indentation(s) or dimple(s) 62 may function to disrupt a flow (e.g., a laminar flow) of fluid flowing through lumen 14 of vessel 12 in such a manner as to cause the flow to begin to or completely switch to a disturbed or turbulent flow distal of distal tip 26 in a manner that may facilitate heat transfer away from vessel wall 16, as discussed above and/or reduce the size of eddy zone 28. Bump(s) 52 and/or dimple(s) 62 may be made of any material. For example, where bump(s) 52 and/or dimple(s) 62 are not located on thermal element 30, bump(s) 52 and/or dimple(s) 62 may be made of a material similar to a material of cap 40 that may be thermally conductive, yet electrically insulating, such that the material may act as a heat sink and facilitate heat transfer away from vessel wall 16.

Alternatively or in addition, another illustrative example may include an elongated shaft 20 having a first portion 20a with a first diameter D1 and a second portion 20b with a second diameter D2, where second portion 20b of elongated shaft 20 may be located at least partially proximal to thermal element 30 and distal tip 26 and/or may be at least partially distal of first portion 20a. Further, one or more fins 54 or one or more vanes 56 may extend outward from second portion 20b in a radial direction, as seen in FIGS. 7A-8B. As fin(s) 54 and vane(s) 56 may extend from second portion 20b, fin(s) 54 and vane(s) 56 may extend from elongated shaft 20 at a position at least partially proximal of thermal element 30 and distal tip 26. Although fin(s) 54 may be generally proximal of thermal element 30, fin(s) 54 may be configured to contact or thermally communicate with thermal element 30 and may extend therefrom toward first portion 20a in at least a substantially straight longitudinal direction, as seen in FIGS. 7A and 7B. Fin(s) 54 may be made from any material allowing, when the function of the material is combined with the contact between thermal element 30 and fin(s) 54, fin(s) 54 to act as a heat sink pulling heat from thermal element 30 and transferring that heat to a fluid passing through vessel lumen 14. For example, fin(s) 54 may be made of a material similar to a material of a cap 40 that is thermally conductive and electrically insulating (e.g., DLC, parylene or other chemical vapor deposited poly(p-xylylene polymers, ceramic materials, highly filled polymers or other similar polymers or other material having similar properties) such that fin(s) 54 may efficiently transfer heat from thermal element 30 to a fluid flowing through vessel lumen 14 due to a relatively large surface area created by the configuration of fin(s) 54.

In addition, vane(s) 56 may extend in a diagonal and generally longitudinal direction and/or helical direction about elongated shaft 20, as seen in FIGS. 8A and 8B, and may be similar to a low-pitched propeller or a helical screw. Optionally, vane(s) 56, like fin(s) 54, may contact or thermally communicate with thermal element 30 and extend therefrom. Although vane(s) 56 may be made from any material, vane(s) 56 may be made from a material similar to the material of cap 40 that may be thermally conductive and electrically insulating or a material with different properties. Thus, vane(s) 56, like fin(s) 54, may also act as a heat sink transferring heat from thermal element 30 to a fluid flowing through vessel lumen 14. Alternatively or in addition to extending from second portion 20b, fin(s) 54 and vane(s) 56 may extend from thermal element 30 and/or cap 40 and/or first portion 20a of elongated shaft 20. Vane(s) 56 may cause a swirl of blood, or another acceleration, that may disturb or thin boundary layers while increasing heat transfer from, or the cooling of vessel wall 16. Alternatively and as may bump(s) 52 and dimple(s) 62, fin(s) 54 and vane(s) 56 may disrupt a flow of fluid flowing through lumen 14 of vessel 12 in such a manner as to cause the flow to begin to or completely switch to a disturbed or turbulent flow distal of distal tip 26 in a manner that may facilitate heat transfer away from vessel wall 16, as discussed above.

Further, alternatively or in addition to using bump(s) 52, dimple(s) 62, fin(s) 54 and/or vane(s) 56, distal end 22 of elongated shaft 20, cap 40 and/or thermal element 30 may include a helical protrusion 58, as seen in FIGS. 10A and 10B and/or a helical channel 60, as seen in FIGS. 9A and 9B. As may the other features extending from or into elongated shaft 20, thermal element 30 and/or cap 40, helical protrusion 58 and helical channel 60 may disrupt a flow of fluid flowing through lumen 14 of vessel 12 in such a manner as to cause the flow to begin to or completely switch to a disturbed or turbulent flow distal of distal tip 26 in a manner that may facilitate heat transfer away from vessel wall 16, as discussed above.

As mentioned, catheter 10 may be utilized in perivascular renal nerve denervation or ablation techniques or other denervation or ablation procedures. For example, catheter 10 may be utilized by inserting distal tip 26 at distal end 22 of elongated shaft 20 of catheter 10 into lumen 14 of vessel 12 (e.g., a renal artery, a renal vein, or, generally, a vessel of a vascular system). Where distal end 22 of elongated shaft 20 may include thermal element 30, tissue 18 adjacent vessel 12 (e.g., a perivascular renal nerve, ganglia, or other nerve or perivascular tissue) may be thermally heated through use of an energy field with RF heating or ultrasound heating or another heating technique to burn or affect tissue 18 (e.g., to denervate tissue 18). Where catheter 10 may include cap 40 or the thin layer, a desired temperature profile may be located between 0.5 millimeters and 5 millimeters into and/or through vessel wall 16. For example, a desired temperature profile may exist at between 1 and 2 millimeters into vessel wall 16 when cap 40 or the thin layer is utilized. As catheter 10 may be configured to have cap 40 (tapered or untapered), bump(s) 52, fin(s) 54, vane(s) 56, spiral protrusion(s) 58, spiral channel(s) 60, indentation(s) or dimple(s) 62 and/or other feature(s) extending from or into elongated shaft 20 that may be able to modify a fluid flowing through lumen 14 of vessel 12, catheter 10 may be configured to remove heat from (e.g., cool) wall 16 of lumen 14 of vessel 12 and may be configured to minimize heat at a luminal surface or vessel wall 16. For example, the disclosed features and configurations of the catheter 10 (e.g., configuration of cap 40, trip feature 32, fins 54, vanes 56, or other surface features altering fluid flow past thermal element 30) may reduce the temperature of the thermal heat zone at vessel wall 16 just distal of the distal tip 26 of catheter 10. In some instances, the zone at vessel wall 16 just distal of the distal tip 26 may experience increase less than 10° C. during the ablation procedure while still providing sufficient heating of desired tissue to provide a therapeutic effect. As a result, due to particular configurations of catheter 10, heat damage to wall 16 during use of catheter 10 may be mitigated or eliminated.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. An ablation catheter apparatus for inserting into a lumen of a vessel having a vessel wall, comprising:
an elongated shaft having a distal end and a proximal end;
a thermal element located at the distal end of the elongated shaft;
wherein the distal end of the elongated shaft is configured to facilitate heat transfer away from the vessel wall.

2. The catheter apparatus of claim 1, further comprising:
a cap extending around a distal tip of the elongated shaft, and
wherein the cap facilitates heat transfer from the vessel wall.

3. The catheter apparatus of claim 2, wherein the cap is tapered.

4. The catheter apparatus of claim 3, wherein a tapered portion of the distal end of the elongated shaft tapers toward the distal tip, and
wherein the cap tapers toward the distal tip and covers at least a section of the tapered portion of the distal end of the elongated shaft.

5. The catheter apparatus of claim 4, further comprising:
a trip feature located on the elongated shaft upstream of the thermal element.

6. The catheter apparatus of claim 2 wherein the cap is made from a material that is thermally conductive and electrically insulating.

7. The catheter apparatus of claim 2, wherein the cap creates a space between a surface of the thermal element and the vessel wall.

8. The catheter apparatus of claim 1, further comprising:
a distal tip located at the distal end of the elongated shaft, and
wherein the thermal element extends from the distal tip of the elongated shaft in a direction upstream of the distal tip.

9. The catheter apparatus of claim 1, wherein the thermal element is an electrode.

10. The catheter apparatus of claim 1, further comprising:
a sensor located at a distal tip of the distal end of the elongated shaft and downstream of the thermal element.

11. The catheter apparatus of claim 1, wherein the distal end of the elongated shaft is configured to create a disturbed or turbulent flow distal a distal tip of the elongated shaft to facilitate heat transfer away from the vessel wall.

12. The catheter apparatus of claim 11, further comprising:
a bump positioned at the distal end of the elongated shaft and adjacent the thermal element.

13. The catheter apparatus of claim 11, further comprising:
a dimple positioned at the distal end of the elongated shaft and adjacent the thermal element.

14. The catheter apparatus of claim 1, wherein the elongated shaft has a first portion with a first diameter and a second portion with a second diameter, where the second diameter is smaller than the first diameter.

15. The catheter apparatus of claim 14, further comprising:
a vane extending from the second portion of the elongated shaft at a position proximal of at least a portion of the thermal element to facilitate heat transfer away from the vessel wall.

16. The catheter apparatus of claim 14, further comprising:
a fin extending from the second portion of the elongated shaft at a position proximal of at least a portion of the thermal element to facilitate heat transfer away from the vessel wall.

17. The catheter apparatus of claim 1, further comprising:
a cap circumferentially extending around the distal end of the elongated shaft to facilitate heat transfer away from the vessel wall.

18. The catheter apparatus of claim 1, wherein the distal end of the elongated shaft is configured to disrupt boundary layers of a fluid flowing through the lumen of the vessel when the distal end of the elongated shaft is inserted into the lumen.

19. A catheter apparatus for cooling a vessel wall, comprising:
an elongated shaft having a distal end and a proximal end, where the distal end is configured to be inserted into a vessel lumen; and
a thermal element located at the distal end of the elongated shaft, and
wherein the distal end of the elongated shaft is configured to modify a flow of fluid through the vessel lumen when the distal end of the elongated shaft is inserted into the vessel lumen to facilitate cooling the vessel wall adjacent a distal tip of the elongated shaft.

20. A method of cooling a wall of a vessel lumen, comprising:
inserting an elongated shaft of a catheter into the vessel lumen, where the elongated shaft includes a distal end and a proximal end;
thermally heating a tissue adjacent the vessel lumen with a thermal element located at the distal end of the elongated shaft; and
removing heat from the wall of the vessel lumen through a configuration of the distal end of the elongated shaft.
